(19) United States
(12) Patent Application Publication
(10) Pub. No.: US 2010/0145053 A1
(43) Pub. Date: Jun. 10, 2010

(54) MODIFIED RELEASE CLOPIDOGREL FORMULATION

(75) Inventors: Pankaj Ramanbhai Patel, Ahmedabad (IN); Sunilendu Bhushan Roy, Ahmedabad (IN); Jay Shantilal Kothari, Ahmedabad (IN)

Correspondence Address: IPHORGAN, LTD., 1130 LAKE COOK ROAD, SUITE 240, BUFFALO GROVE, IL, 60089, US

(73) Assignee: CADILA HEALTHCARE LIMITED, Ahmedabad, Gujarat (IN)

(21) Appl. No.: 12/295,932

(22) PCT Filed: Apr. 3, 2007
(86) PCT No.: PCT/IN07/00142

§ 371 Date: Feb. 23, 2010

(30) Foreign Application Priority Data
Apr. 5, 2006 (IN) 528/MUM/2006

(51) Int. Cl. C07D 495/04

(52) U.S. Cl. 546/114

(57) ABSTRACT

A once daily dosage form comprising Clopidogrel or a salt of Clopidogrel equivalent to 50 mg to 150 mg Clopidogrel is disclosed. Said dosage form provides at least one of the following in vivo plasma profile of Clopidogrel selected from:
(a) Mean Tmax of about 3 or more hours.
(b) Mean Cmax not above 1000 picogram/ml.
(c) Mean AUC₀₋₄₉ₕ of more than 2500 picogram/ml/hr.

### BACKGROUND OF THE INVENTION

1. Field of the Invention

The present invention relates to pharmaceutical composition of Clopidogrel and/or its pharmaceutically acceptable salts/solvates. Particularly, the present invention relates to the modified release formulations of Clopidogrel and/or its pharmaceutically acceptable salts/solvates. The present invention further provides a process for preparing modified release formulations of Clopidogrel and/or its pharmaceutically acceptable salts/solvates thereof. The present invention further provides once daily dosage form comprising Clopidogrel or a salt of Clopidogrel equivalent to 50 mg to 150 mg Clopidogrel, said dosage form providing plasma profile wherein the Mean Tmax is of about 3 or more hours and Mean Cmax is not above 1000 picogram/ml for Clopidogrel. The formulations of present invention of Clopidogrel provide substantially lower levels of inactive Clopidogrel carboxy acid metabolite as compared to Brand product, Plavix®, Clopidogrel tablets 75 mg marketed by Sanofi.

Clopidogrel is chemically known as Methyl (+)-(S)-alpha-(o-chlorophenyl)-6,7-dihydrothieno(3,2-c)pyridine-5(4H)-acetate and represented by following structure of formula (I):

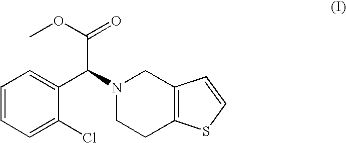

2. Background Art

Clopidogrel is an inhibitor of platelet aggregation. A variety of drugs that inhibit platelet function have been shown to decrease morbid events in people with established cardiovascular atherosclerotic disease as evidenced by stroke or transient ischemic attacks, myocardial infarction, unstable angina or the need for vascular bypass or angioplasty. This indicates that platelets participate in the initiation and/or evolution of these events and that inhibiting them can reduce the event rate.

Clopidogrel selectively inhibits the binding of adenosine diphosphate (ADP) to its platelet receptor and the subsequent ADP-mediated activation of the glycoprotein GPIIb/IIIa complex, thereby inhibiting platelet aggregation. Biotransformation of Clopidogrel is necessary to produce inhibition of platelet aggregation. Clopidogrel also inhibits platelet aggregation induced by agonists other than ADP by blocking the amplification of platelet activation by released ADP. Clopidogrel does not inhibit phosphodiesterase activity.

Clopidogrel acts by irreversibly modifying the platelet ADP receptor. Consequently, platelets exposed to Clopidogrel are affected for the remainder of their lifespan.

Dose dependent inhibition of platelet aggregation can be seen 2 hours after single oral doses of Clopidogrel. Repeated doses of 75 mg Clopidogrel per day inhibit ADP-induced platelet aggregation on the first day, and inhibition reaches steady state between Day 3 and Day 7. At steady state, the average inhibition level observed with a dose of 75 mg Clopidogrel per day was between 40% and 60%. Platelet aggregation and bleeding time gradually return to baseline values after treatment is discontinued, generally in about 5 days.

Clopidogrel is available in the market in the form of crystalline bisulfate salt namely Clopidogrel bisulfate and sold under Brand name Plavix®.

The therapeutic application of Clopidogrel as blood-platelet aggregation inhibiting agents and antithrombotic agent and its preparation is disclosed in U.S. Pat. No. 4,529,596.

U.S. Pat. No. 6,429,210 disclosed a crystalline form of Clopidogrel hydrogen sulfate designated as form II. The process described in EP 281459 for the preparation of Clopidogrel hydrogen sulfate leads to a crystalline form (form 1). In the marketed product, Plavix® crystalline Clopidogrel hydrogen sulfate was used in pharmaceutical compositions.

US2006/0264636 discloses new salts of Clopidogrel viz. Clopidogrel mesylate, Clopidogrel besylate and Clopidogrel tosylate, methods for their preparation and pharmaceutical compositions containing them.

US2007/0048370 disclose formulation of Clopidogrel or the solvates or hydrates thereof, with the proviso that the salt is not Clopidogrel hydro-iodide, wherein the tablet contains no ionic and/or basic tabletting excipient, and no polyethylene glycol 6000.

Clopidogrel is a prodrug. The active metabolite, a thiol derivative, is formed by oxidation of Clopidogrel to 2-oxo-Clopidogrel and subsequent hydrolysis. The oxidative step is regulated primarily by Cytochrome P₄₅₀ isoenzymes 2B6 and 3A4 and to a lesser extent by 1A1, 1A2 and 2C19. The active thiol metabolite, which has been isolated in vitro, binds rapidly and irreversibly to platelet receptors, thus inhibiting platelet aggregation. This metabolite has not been detected in plasma.

The kinetics of the main circulating metabolite were linear (plasma concentrations increased in proportion to dose) in the dose range of 50 to 150 mg of Clopidogrel. Ref: IPHA Electronic Medicines compendium, dated 15 Sep., 2006.

Although platelets lack nuclei and are the smallest circulating human cells, they play an integral and complex role in the process of thrombosis, both physiological and patho-physiological. Activation and aggregation of platelets play a central role in the propagation of intracoronary thrombi after (1) spontaneous atherosclerotic plaque disruption that results in myocardial ischemia or infarction in the acute coronary syndromes (ACS), or (2) the mechanical disruption that results from percutaneous coronary intervention (PCI). Platelets initially adhere to collagen and von Willebrand factor at the site of the disrupted plaque, resulting in an initial platelet monolayer. After activation, platelets release secondary agonists such as thromboxane A₂ and adenosine diphosphate (ADP), which in combination with thrombin generated by the coagulation cascade result in stimulation and recruitment of additional platelets. With this patho-physiological background, it is not surprising that antiplatelet therapy is a cornerstone of the management of patients with ACS, especially those undergoing PCI.

Studies have shown a dose- and time-dependent variability in response to Clopidogrel as measured by optical platelet aggregometry in response to ADP. In a study by Gurbel et al, 96 patients undergoing elective coronary stenting were monitored before and at multiple time points after standard Clopidogrel therapy (300-mg loading dose followed by 75 mg daily).

Clopidogrel resistance, empirically defined as <10% reduction in aggregation in response to 5 μmol/L ADP compared with pretreatment values, was seen in 63% of patients at 2 hours, 31% at 24 hours, 31% at 5 days, and 15% at 30 days.

Patients with the highest pretreatment values had the least antithrombotic protection over the first 5 days. In another report, Muller et al defined nonresponders as those with <10% reduction in platelet aggregation to ADP and semiresponders as those with 10% to 29% reduction 4 hours after 600-mg Clopidogrel load, as no additional effect was seen with this treatment regimen at 24 hours. This study found that to 5 μmol/L ADP, 5% were nonresponders and 9% were semiresponders, and to 20 μmol/ADP, 11% were nonresponders and 26% were semiresponders. Although not designed to evaluate clinical outcomes, an intriguing finding in the Muller study was that 2 patients (of 105 tested) developed subacute stent thrombosis, and both met the definition of Clopidogrel nonresponse. An additional report correlated anginal class to platelet inhibition and found that patients with higher anginal class on presentation had less inhibition of platelet aggregation after loading with 450 mg of ADP.

Several mechanisms of Clopidogrel resistance are possible. Extrinsic mechanisms include inappropriate dosing or underdosing of Clopidogrel and drug-drug interactions, including a possible interaction between Clopidogrel and atorvastatin. There is a positive correlation of Clopidogrel response with CYP3A4 activity (measured by erythromycin breath test), suggesting that an important mechanism may be variable conversion to the active metabolite. Other potential extrinsic mechanisms could include variable absorption of the prodrug or clearance of the active metabolite. Intrinsic mechanisms could include P2Y₁₂ receptor variability, increase in number of receptors, increased release of ADP, or upregulation of other platelet activation pathways. Ref: Circulation 2004;109:3064-3067, American Heart Association, Inc.

Recently, new clinical data has emerged to show that standard doses of Plavix do not provide adequate platelet inhibition in about 25% of patients. There are several possible explanations for Plavix resistance and it is still not clear whether Plavix resistance is a real phenomenon or an outcome of ineffective dosing. Recent studies have suggested that Plavix “resistance” can be substantially diminished through a higher loading dose. This issue of “Plavix resistance” is gaining more attention among cardiologists, and a study published in Circulation in 2004 showed that patients receiving the least benefit from Plavix [as determined by adenosine diphosphate (ADP)-induced percentage change in platelet aggregation] suffered much higher cardiovascular events risk. Ref: Circulation. 2004;109:3171-3175.) 2004 American Heart Association, Inc.

Platelet aggregation is an important part of the thrombus formation process. This platelet aggregation phenomena initiated by Adenosine 5-di phosphate, epinephrine & various platelets factors are extruded from platelet granules & formation of thromboxanes which play a major role in platelet aggregation. Agents capable of interfering with platelet recruitment into forming thrombus can play an important role in the treatment of atherosclerosis, thrombosis and acute coronary syndromes. Measurement of platelet aggregation in vivo in platelet rich plasma obtained from animals is a fundamental tool in platelet studies. ADP is an aggregating agent for in vitro/ex vivo screening of normal platelet function represents as an important diagnostic tool for identifying the causes of bleeding disorders.

It is reported that Clopidogrel bisulfate dosage provides a peak concentration of Clopidogrel and its active metabolite in 1-3 hours post dosing. This would provide high concentration of active metabolite immediately after dosing and lower concentration at 24 hours post dosing. Moreover, 85% of Clopidogrel after absorption is inactivated by esterases in the blood and only 15% is available to undergo hepatic conversion to the active metabolite. In summary the patient is exposed to bleeding risk at peak levels at steady state and poor platelet inhibition at trough levels at steady state.

More recently, various research groups and clinicians have reported emerging issue of Clopidogrel resistance. This may increases the risk of recurrent cardiovascular events. The inventors of the present invention have surprising found that a modified release formulation of Clopidogrel and its pharmaceutically acceptable salts may address issue of Clopidogrel resistance thereby improving response rate. In addition, a modified release formulation also offers potential to improve safety, convenience and compliance with Clopidogrel therapy. Overdose following Clopidogrel administration may lead to prolonged bleeding time and subsequent bleeding complications. Appropriate therapy should be considered if bleedings are observed. In sum, a modified release formulation of Clopidogrel will improved care for millions of people in need of better antiplatelet therapy.

The present invention meets the unfulfilled needs of the pharmaceutical industry.

The modified release may be such as a delayed, extended, pulsed or sustained release. The modification of the release may be desired for a number of reasons, such as for minimizing the side effects of the drug such as gastric bleeding or to prevent development of Clopidogrel resistance in patients requiring life-long treatment with Clopidogrel.

### SUMMARY OF THE INVENTION

One of the important objects of the present invention is to provide a modified release pharmaceutical composition of Clopidogrel or its pharmaceutically acceptable salts/solvates.

Another object of the present invention is to provide a modified release pharmaceutical composition of using Clopidogrel or its pharmaceutically acceptable salts/solvates equivalent to 50 mg to 150 mg Clopidogrel providing suitable plasma profiles with Cmax not above 1000 picogram/ml and Tmax more than 3 hours.

Yet another object of the invention is to provide formulations of Clopidogrel that provide substantially lower levels of inactive Clopidogrel carboxy acid metabolite as compared to Brand product, Plavix®, Clopidogrel tablets 75 mg marketed by Sanofi.

Yet another object of the present invention is to provide processes for manufacturing modified release formulations of Clopidogrel or its pharmaceutically acceptable salts/solvates.

The above an other objects of the present invention are achieved by providing a once daily dosage form comprising Clopidogrel or a salt of Clopidogrel equivalent to 50 mg to 150 mg Clopidogrel, said dosage form providing at least one of the following in vivo plasma profile of Clopidogrel selected from:
(a) Mean Tmax of about 3 or more hours.
(b) Mean Cmax not above 1000 picogram/ml.
(c) Mean AUC₀₋₄₉ₕ of more than 2500 picogram/ml/hr.
Preferably, the once daily dosage form comprises Clopidogrel or a salt of Clopidogrel equivalent to 50 mg to 150 mg Clopidogrel, said dosage form providing an in vivo plasma profile of Clopidogrel carboxy acid metabolite having Mean Cmax not above 1400 ng/ml.
Preferably, the dosage form comprises Clopidogrel or a salt of Clopidogrel equivalent to 75 mg Clopidogrel.
Preferably, the said mean Cmax is less than about 900 picogram/ml.
Preferably, the said mean Cmax is less than about 900 picogram/ml.
Preferably, the said mean Cmax is less than about 800 picogram/ml.
Preferably, the said mean Cmax is not above 1200 ng/ml., more preferably, not more than 1100 ng/ml.

### BRIEF DESCRIPTION OF THE DRAWINGS

FIG. 1: Comparative dissolution profile of Plavix®.

FIG. 2: Comparative platelet aggregation effect of Plavix, Clopidogrel Tablets 75 mg.

FIG. 3: Comparative % aggregate size of platelets in the two formulations Plavix® Vs. Clopidogrel tablets.

FIG. 4: Comparative % aggregate size of platelets in the two formulations Plavix® Vs. Clopidogrel tablets.

FIG. 5: Mean Plasma concentration time profile of Clopidogrel carboxyacid metabolite (ng/mL) following a single dose of Plavix®, Clopidogrel tablets 75 mg.

### DETAILED DESCRIPTION OF THE INVENTION

According to the present invention, Clopidogrel as used here in after is meant to include its salt such as acid addition salts like hydrochloric, hydrobromic, sulfuric, hydrogen bisulfate, nitric, and phosphoric acid; or with an organic acid selected from acetic, propanoic, hydroxyacetic, lactic, pyruvic, oxalic, malonic, succinic, maleic, fumaric, malic, tartaric, citric, methanesulfonic, ethanesulfonic, benzenesulfonic, p-toluenesulfonic, napthane sulfonic, cyclamic, salicylic, p-aminosalicylic, and pamoic acid. In addition, the Clopidogrel can also be used and includes its form of a solvate for example water or organic solvents.

According to the present invention, Clopidogrel as used herein can be in the form of crystalline, partially crystalline or substantially amorphous form.

According to the present invention, Clopidogrel can be used in therapeutic effective amount in patient need thereof.

According to first aspect of the present invention, there is provided a once daily dosage form comprising Clopidogrel or a salt of Clopidogrel equivalent to 50 mg to 150 mg Clopidogrel, said dosage form providing at least one of the following in vivo plasma profile of Clopidogrel selected from: (a) Mean Tmax of about 3 or more hours. (b) Mean Cmax not above 1000 picogram/ml., (c) Mean AUC₀₋₄₉ₕ of more than 2500 picogram/ml/hr.

According to another aspect of the present invention, there is provided a once daily dosage form comprising Clopidogrel or a salt of Clopidogrel equivalent to 50 mg to 150 mg Clopidogrel, said dosage form providing of Clopidogrel carboxy acid metabolite having Mean Cmax not above 1400 ng/ml.

In the preferred embodiment of the invention, wherein the said mean Cmax of Clopidogrel is less than about 800 picogram/ml.

The inventors of the present invention have surprisingly found modified release formulation of Clopidogrel can deliver better consistent inhibition of platelet aggregation.

According to another aspect of the present invention, there is provided a modified release formulations of Clopidogrel comprising Clopidogrel and at least one rate-controlling polymer. Rate controlling polymer is either hydrophilic or hydrophobic in nature. Mixture of the rate controlling material or more is also used to provide formulations of present invention.

In the preferred embodiment, modified formulation of Clopidogrel is in the form of a matrix comprising Clopidogrel, one or more hydrophilic polymers, optionally an enteric polymer and pharmaceutically acceptable excipients. The formulations of the present invention can be in the form of oral dosage forms such as tablets, capsules, pellets, granules, microtablets etc. Preferably, formulation is either compressed into tablets or granulated and filled into capsules.

Clopidogrel used in the present invention can be in the range of 5 to 50% weight of the composition. Rate controlling polymer used in the present invention can be in the range of 10 to 90% weight of the composition

Suitable hydrophilic polymers include cellulose ethers such as hydroxypropyl methyl cellulose, hydroxypropyl ethyl cellulose, hydroxy ethylcellulose, hydroxypropylcellulose, or other water soluble or swellable polymers such as sodium carboxymethyl cellulose, locust bean gum, xanthan gum, acacia, tragacanth gum, guar gum, karaya gum, alginates, gelatin, albumin, carbomers, alginates, polyvinyl pyrollidones or mixtures thereof. These hydrophilic polymers also include polyacrylate polymers, such as homopolymers based on acrylic acid cross-linked with allyl sucrose or allyl pentaerythritol, or copolymers based on acrylic acid and long chain (C₁₀-C₃₀) allyl acrylates cross-linked with allylpentaerythritol. The polyacrylate polymers may be used alone or in admixture with cellulose ethers such as methylcellulose, hydroxypropyl methylcellulose, hydroxypropylcellulose, hydroxyethylcellulose, and the like. According to the invention, the hydrophilic polymers are present in amounts ranging from about 10% to about 70% by weight of the system.

The preferred hydrophilic polymers are selected from the group consisting of cellulose ethers such as hydroxypropyl methylcellulose, hydroxypropylcellulose, methylcellulose and mixtures thereof.

According to a preferred embodiment of the present invention, the hydrophilic polymer is a mixture of a hydroxypropyl methyl cellulose whose 2% by weight aqueous solution has a viscosity greater than 10,000 cps, and hydroxypropylcellulose whose 2% by weight aqueous solution has a viscosity less than 5000 cps. The hydroxypropyl methylcellulose may be present in amounts from about 5% to 70% by weight, preferably from about 20% to 45% by weight, of the total weight of the system.

The hydroxypropylcellulose may be present in amounts from about 5% to 50% by weight, preferably from about 15% to 25% by weight, of the system.

Examples of hydroxypropyl methyl cellulose polymers that may be used in the present invention include Methocel such as, Methocel K4M®, Methocel K15M®, Methocel K100M®, Methocel K100LV® and the like. Hydroxypropylcellulose polymers that may be used in the present invention include, for example, HPC such as HPC-L, HPC-M, Klucel® such as Klucel GF®, Klucel JF®, Klucel HF®, Klucel HX®F and the like.

The enteric polymers that may be used in the present invention include polyacrylate copolymers such as Methacrylic Acid Copolymer, cellulose derivatives, such as cellulose acetate phthalate, hydroxypropyl methylcellulose phthalate, hydroxypropyl methylcellulose acetate succinate; and polyvinyl acetate phthalate and the like.

Preferably enteric polymer may be Polymethacrylate. Polymethacrylate can be a cationic, anionic or neutral polymer or copolymer on the basis of monomers having a methacrylic moiety. Mixtures of such polymers or copolymers can also be used.

Preferably, the polymethacrylate is a polymer or copolymer based on at least one of dimethylaminoethyl methacrylates, methacrylic acid and methacrylic acid esters. It is particularly preferred that the polymethacrylate is a methacrylic acid copolymer or a copolymer of dimethylaminoethyl methacrylates and methacrylic acid esters.

Various different types of polymethacrylates are commercially available e.g. under the trade name Eudragit. A preferred material for use in the composition according to the invention is Eudargit L®, which is a methacrylic acid copolymer or Eudragit EPO. Particularly preferred is Eudragit EPO®

According to another embodiment of the present invention, the enteric polymer can be a polyacrylate enteric polymer. The enteric polymer may be present in amounts from about 0.5% to 30% by weight of the system.

In the preferred embodiment of the preset invention, Clopidogrel is dispersed in hydrophilic polymers and the release of the drug is controlled primarily by diffusion of the drug, or by surface erosion of the hydrophilic polymers into the surrounding medium, or by a combination of the two processes achieved by the methods known to the person skilled in the art.

According to another aspect of the present invention, there is provided a modified or extended release formulations comprising Clopidogrel, hydrophobic rate-controlling polymer alone or mixture of hydrophobic and hydrophilic rate-controlling polymer. Hydrophobic rate-controlling polymer is either incorporated into or coated onto dosage form to provide modified release formulations. Without any limitation, it is preferred that when hydrophobic rate-controlling polymer is used alone it is incorporated into dosage forms and when it is used along with hydrophilic rate-controlling polymer it is coated onto dosage form to provide modified release formulations.

The most preferred hydrophobic rate-controlling polymer is selected from ethyl cellulose, polymethacrylates and mixtures thereof. Most preferred polymethacrylates are poly ethyl acrylate, poly methyl methacrylate, poly trimethylaminoethyl methacrylates and mixtures thereof.

According to another embodiment of the present invention, modified release formulation of Clopidogrel further comprises pharmaceutically acceptable excipients.

Pharmaceutically acceptable excipients includes and can be selected from any of disintegrants, diluents, stabilizers, binder, lubricants, glidents, coloring agents, stabilizers, masking agents, surfactants, solubilizers, moisture scavengers, antioxidants, buffering agent, adsorbents, adhering agents and the like.

Disintegrants can be selected from calcium phosphate, (mono/di/tri basic), carboxy methyl cellulose salt such as calcium, sodium; cellulose, microcrystalline cellulose, chitosan, silicone dioxide, croscarmellose sodium, Cross-linked polyvinyl pyrrolidone used in the present invention is commercially available under the trade name Crospovidone, guar gum, hydroxypropyl cellulose, magnesium aluminum silicate, methyl cellulose, micro crystalline cellulose, povidone, sodium starch glycolate, starch or mixtures thereof or the well known disintegrants known by the person skilled in the art.

Diluents can be selected from calcium carbonate, calcium phosphate, calcium sulfate, cellulose, cellulose acetate, dextrates, dextrines, dextrose, ethyl cellulose, microcrystalline cellulose, polydextrose, polymethacrylates, sucrose, lactose, starch, mannintol or mixtures thereof or the well known Diluents known by the person skilled in the art.

Binder can be selected from acacia, alginic acid, carbomers, carboxymethyl cellulose sodium, chitosan, dextrates, dextrins, dextrose, ethyl cellulose, gelatin, glucose, guar gum, hydroxyethyl cellulose, hydroxyethylmethyl cellulose, hydroxy propyl cellulose, hydroxypropyl methyl cellulose, hypromellose, magnesium silicate, aluminum silicate, maltodextrin, maltose, micro crystalline cellulose, poloxamer, polydextrose, polyethylene oxide, povidone, sodium alginate, starch, sucrose or mixtures thereof or the well known binder known by the person skilled in the art.

Stabilizers can be selected from Sodium citrate, NaCl, K₂HPO₄, Meglumine, Sodium ascorbate, KCl, Sodium sulfite, Poloxamer 188/407, Polyethylene glycol, glyceryl monooleate, alginic acid, albumin, ammonium alginate, ascorbic acid, ascorbyl palmitate, bentonite, butylated hydroxytolune, calcium alginate, calcium state, carboxymethylcellulose calcium, carboxymethylcellulose sodium, carrageenan, ceratonia, colloidal silicon dioxide, cyclodextrins, diethanolamine, edetates, ethylene glycol palmisterate, glycerin monosterate, guargum, magnesium aluminum silicate, lecithin, hypromellose, hydroxy propyl cellulose, polacrilin potassium, pectin, poloxamer, polyvinyl alcohol, propyl gallate, propylene glycol, xylitol, zinc acetate, raffinose, sodium borate, trehalose, propylene glycol alginate, sulfobutylether beta-cyclo dextrin or mixtures thereof or the well known stabilizers know by the person skilled in the art.

Glidants can be selected from the group consisting of silicon dioxide, talc, Calcium stearate, Magnesium stearate and Aluminium stearate, or mixtures thereof.

Lubricants can be selected from magnesium stearate, magnesium lauryl sulfate, sodium lauryl sulfate, sodium stearyl fumarate, calcium stearate, microcrystalline cellulose and silicone dioxide, hydrogenated cottonseed oil, hydrogenated castor oil, stearic acid, zinc stearate or mixtures thereof.

Buffering agents can be selected from ammonia solution, calcium carbonate, calcium phosphate, citric acid, sodium phosphate, diethanol amine, malic acid, monosodium glutamate, phosphoric acid, potassium citrate, sodium acetate, sodium bicarbonate, sodium borate, sodium citrate, sodium hydroxide, sodium lactate, triethanol amine or mixtures thereof or the well known buffering agent known by the person skilled in the art.

The modified release formulation of Clopidogrel can be prepared by employing any method selected from wet granulation, dry granulation, melt granulation or the method known by the person skilled in the art.

In one embodiment of the aspect of present invention, Clopidogrel alone or optionally with other excipients is blended with polymer and compressed into tablets.

Alternatively the blend is compacted to produce granules, which are further compressed into tablets.

In another embodiment, polymer is dissolved or dispersed in suitable solvent and this solution or dispersion is used to granulate Clopidogrel alone or Clopidogrel blended with other excipients. In this embodiment without any limitation when Clopidogrel is granulated alone, spray coating or fluid bed coatings are preferred whereas when Clopidogrel is blended with excipients and granulated granulation using high shear granulator is preferred.

In yet another embodiment, Clopidogrel cores are prepared by either drug layering onto preformed pellets or alternatively Clopidogrel is blended with suitable excipients and wet granulated. The wet granulate is extruded and spheronised to produce spherical cores of Clopidogrel. The cores prepared by any of the above methods are then coated with ethyl cellulose or mixture of ethyl cellulose along with hydrophilic substance selected from the group consisting of hydroxy propyl methyl cellulose, hydroxy propyl cellulose, hydroxy ethyl cellulose, polyvinyl pyrrolidone, alginates, gums, carbomers or mixtures thereof.

A preferred hydrophobic polymer according to the present invention is ethyl cellulose.

Ethyl cellulose of Viscosity from about 7 cps to about 100 cps is preferred. Amongst these ethyl cellulose of viscosity from about 10 cps to about 50 cps are most preferred. The amount of ethyl cellulose incorporated varies with the grade selected. Amount from about 0.5 weight percent to about 30 weight percent is preferred. The amount from about 5 weight percent to about 15 weight percent is most preferred.

When hydrophobic polymers is used along with hydrophilic rate-controlling polymer, without any limitation, low viscosity grades of hydrophilic substances e.g. hydroxy propyl methyl cellulose from 3 cps to 15 cps, poly vinyl pyrollidone from PVP K-12 to PVPK-90 are preferred.

Another aspect of the invention is to provide modified release formulations of Clopidogrel by use of hydrophobic rate-controlling polymer having lower melting point than that of Clopidogrel. The difference in the melting point of Clopidogrel and low melting hydrophobic rate-controlling polymer should be at least 30° C. This low melting hydrophobic material is melted to fluid consistency and Clopidogrel is dispersed into it until uniformly dispersed. Molten mass is then screened and or dried to produce granules and granules are further compressed into tablets. Here optionally before compression, granules are blended with suitable excipients. The low melting hydrophobic rate-controlling polymer is selected from the group consisting of cetyl alcohol, cetostearyl alcohol, white wax, yellow wax, microcrystalline wax and mixtures thereof.

In yet another embodiment, Clopidogrel is blended with one or more polymers such as hypromellose, Carbopol, povidone, hydroxyethyl cellulose, hydroxy propyl cellulose, ethyl cellulose and other suitable excipients such as fillers/diluents, binders, disintegrants and lubricants and compressed to tablets. Alternately the excipient mixture with Clopidogrel as prepared above may be subjected to dry granulation followed by compression.

The modified release formulation of the present invention may be coated optionally.

The increased compliance observed with the modified release dosage form of Clopidogrel can improve the appropriate management of patients in need to treatment with Clopidogrel including, but not restricted for the reduction of atherosclerotic events (myocardial infarction, stroke, and vascular death) in patients with atherosclerosis documented by recent stroke, recent myocardial infarction, or established peripheral arterial disease.

The modified release formulation of Clopidogrel and its process described in the present invention is demonstrated in examples illustrated below. These examples are provided as illustration only and therefore should not be construed as limitation of the scope of invention.

### Example 1-4

| | % w/w | | | |
|---|---|---|---|---|
| Ingredient | Example 1 | Example 2 | Example 3 | Example 4 |
| Clopidogrel besylate | 24.9 | 24.9 | 24.9 | 24.9 |
| equivalent to | | | | |
| Clopidogrel 75 mg | | | | |
| Microcrystalline | 38.3 | 35.8 | 33.3 | 35.6 |
| cellulose | | | | |
| Hypromellose | 20.0 | 20.0 | 20.0 | 20.0 |
| (Methocel K4M ®) | | | | |
| Carbopol 71 G | 10.0 | 10.0 | 10.0 | 10.0 |
| Crospovidone | 5.0 | 7.5 | 10.0 | 7.5 |
| (Polyplasdone XL ®) | | | | |
| Colloidal silicondioxide | 0.8 | 0.8 | 0.8 | 1.1 |
| (Aerosil 200 ®) | | | | |
| Hydrogenated cottonseed | 1.0 | 1.0 | 1.0 | 1.0 |
| oil (Lubritab ®) | | | | |

Examples 1-4 were prepared by the following procedure:

Clopidogrel besylate, microcrystalline cellulose, hypromellose, carbopol and crospovidone were mixed in diffusion blender. Colloidal silicondioxide and hydrogenated cottonseed oil were added to above mix in the diffusion blender to get lubricated blend. The lubricated blend was compressed to tablets.

### Example 5

| Ingredient | % w/w |
|---|---|
| Clopidogrel besylate equivalent to Clopidogrel 75 mg | 22.4 |
| Microcrystalline cellulose | 34.6 |
| Carbopol 71 G | 10.0 |
| Polyethylene oxide (PEO WSR 303 ®) | 20.0 |
| Crospovidone (Polyplasdone XL ®) | 10.0 |
| Colloidal silicondioxide (Aerosil 200 ®) | 2.0 |
| Hydrogenated cottonseed oil (Lubritab ®) | 1.0 |

Example 5 was prepared by the following procedure:

Clopidogrel besylate, microcrystalline cellulose, polyethylene oxide, carbopol and crospovidone were mixed in diffusion blender. Colloidal silicondioxide and hydrogenated cottonseed oil were added to above mix in the diffusion blender to get lubricated blend. The lubricated blend was compressed to tablets with average weight of 500 mg.

### Example 6-7

| | % w/w | |
|---|---|---|
| Ingredient | Example 6 | Example 7 |
| Clopidogrel besylate equivalent to | 24.9 | 24.9 |
| Clopidogrel 75 mg | | |
| Microcrystalline cellulose | 53.1 | 63.1 |
| Povidone K 90 | 20.0 | 10.0 |
| Colloidal silicondioxide (Aerosil 200 ®) | 1.0 | 1.0 |
| Magnesium stearate | 1.0 | 1.0 |

Examples 6-7 were prepared by the following procedure:

Clopidogrel besylate, microcrystalline cellulose and povidone were mixed in diffusion blender. Colloidal silicondioxide and magnesium stearate were added to above mix in the diffusion blender to get lubricated blend. The lubricated blend was compressed to tablets with average weight of 450 mg.

### Example 8

| Ingredient | % w/w |
|---|---|
| Clopidogrel besylate equivalent to Clopidogrel 75 mg | 38.6 |
| Lactose (Pharmatose DCL 21) | 31.9 |
| Hypromellose (Methocel K4M ®) | 17.3 |
| Carbopol 71 G | 8.1 |
| Colloidal silicondioxide (Aerosil 200 ®) | 1.6 |
| Hydrogenated cottonseed oil (Lubritab ®) | 1.0 |
| Talc | 1.6 |

Example 8 was prepared by the following procedure:

Clopidogrel besylate, lactose, hypromellose and carbopol were mixed in diffusion blender. Colloidal silicondioxide, hydrogenated cottonseed oil and talc were added to above mix in the diffusion blender to get lubricated blend. The lubricated blend was compressed to tablets with average weight of 290 mg.

### Example 9

| Ingredient | % w/w |
|---|---|
| Clopidogrel besylate equivalent to Clopidogrel 75 mg | 24.9 |
| Microcrystalline cellulose | 53.1 |
| Povidone K90 | 20.0 |
| Colloidal silicondioxide (Aerosil 200 ®) | 1.0 |
| Zinc stearate | 1.0 |

Example 9 was prepared by the following procedure:

Clopidogrel besylate, Povidone 90, microcrystalline cellulose and colloidal silicondioxide were sifted through #40. Zinc stearate was sifted through #60. Sifted zinc stearate was added to remaining sifted excipients and blended in double cone blender for 10 mins. The lubricated blend was slugged using Roller compactor at hardness 4-6 kp. The slugs were deslugged using Oscillating granulator through screen #20. Extra-granular Povidone 90 and colloidal silicondioxide were sifted through #30 and added to sized granules and blended. Zinc stearate was sifted through #60 blended with above mix to get lubricated blend. Lubricated blend was compressed to tablets with average weight of 450 mg.

### Example 10

| Ingredient | % w/w |
|---|---|
| Clopidogrel besylate equivalent to Clopidogrel 75 mg | 24.9 |
| Microcrystalline cellulose | 53.1 |
| Povidone K90 | 20.0 |
| Colloidal silicondioxide (Aerosil 200 ®) | 1.0 |
| Zinc stearate | 1.0 |

Example 10 was prepared by the following procedure:

Clopidogrel besylate, microcrystalline cellulose and povidone were mixed in diffusion blender. Colloidal silicondioxide and zinc stearate were added to above mix in the diffusion blender to get lubricated blend. The lubricated blend was compressed to tablets with average weight of 450 mg.

### Example 11

| Ingredient | % w/w |
|---|---|
| Clopidogrel bisulphate equivalent to Clopidogrel 75 mg | 27.96 |
| Microcrystalline cellulose | 49.04 |
| Povidone K90 | 20.00 |
| Colloidal silicondioxide (Aerosil 200 ®) | 1.50 |
| Zinc stearate | 1.50 |

Example 11 was prepared by the following procedure:

Clopidogrel bisulphate, microcrystalline cellulose and povidone were mixed in diffusion blender. Colloidal silicondioxide and zinc stearate were added to above mix in the diffusion blender to get lubricated blend. The lubricated blend was compressed to tablets with average weight of 350 mg.

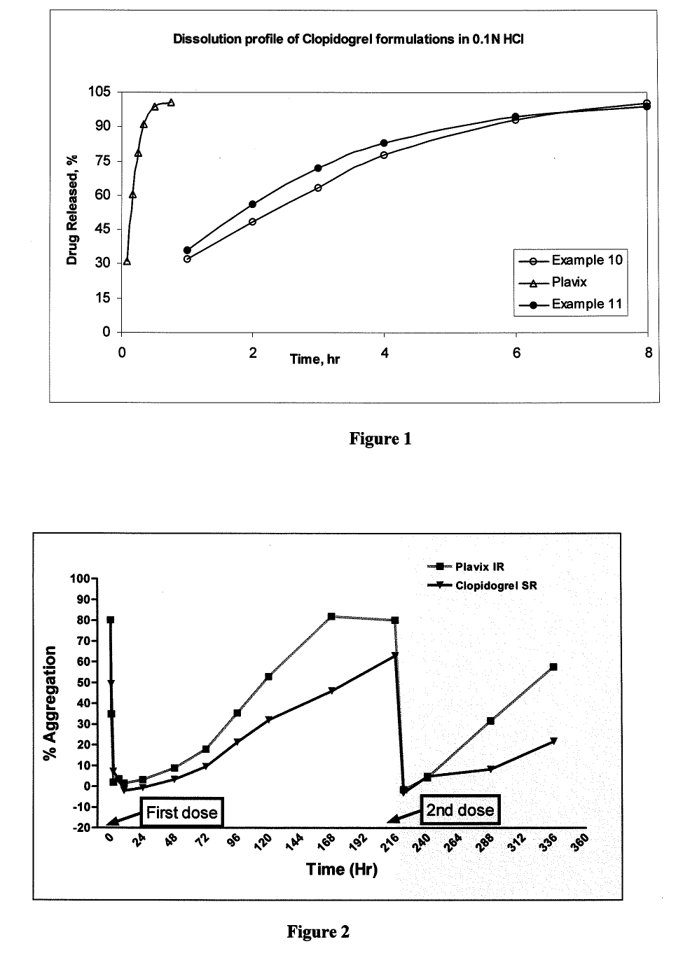

The tablets, of Examples 1-11 were tested for dissolution profile in 900 ml 0.1N HCl using USP Type II apparatus at 50 rpm up to 8 hrs. Dissolution profile of tablets prepared as per example 1-11 and Plavix® is tabulated below in Table 1 & 2. FIG. 1 depicts comparative dissolution profile of Plavix®, Example 10 & 11 in 900 ml 0.1N HCl using USP Type II apparatus at 50 rpm up to 8 hrs.

TABLE 1
Comparative dissolution profile of Plavix ®, Clopidogrel Tablets 75 mg,
Example 1-5 in 900 ml 0.1N HCl, USP Type II apparatus at 50 rpm.

| | % Drug | | | | | | |
|---|---|---|---|---|---|---|---|
| Time | released | Time | % Drug released | | | | |
| in hrs | Plavix ® | in hrs | Ex. 1 | Ex. 2 | Ex. 3 | Ex. 4 | Ex. 5 |
| 0.08 | 30.9 | 1 | 13.8 | 22.4 | 30.5 | 34.3 | 23.9 |
| 0.17 | 60.2 | 2 | 22.3 | 37.3 | 43.1 | 53.4 | 36.9 |
| 0.25 | 78.4 | 3 | 28.5 | 48.3 | 57.8 | 67.6 | 49.2 |
| 0.33 | 90.9 | 4 | 33.7 | 57.0 | 69.9 | 78.3 | 57.9 |
| 0.50 | 98.5 | 6 | 41.9 | 70.0 | 83.5 | 90.9 | 72.9 |
| 0.75 | 100.6 | 8 | 49.0 | 79.6 | 94.8 | 98.9 | 83.4 |

TABLE 2
Comparative dissolution profile of Plavix ®, Clopidogrel Tablets 75 mg,
Example 6-11 in 900 ml 0.1N HCl, USP Type II apparatus at 50 rpm.

| | % Drug | | | | | | | |
|---|---|---|---|---|---|---|---|---|
| Time | released | Time | % Drug released | | | | | |
| in hrs | Plavix ® | in hrs | Ex. 6 | Ex. 7 | Ex. 8 | Ex. 9 | Ex. 10 | Ex. 11 |
| 0.08 | 30.9 | 1 | 25.7 | 28.6 | 24.5 | 27.3 | 31.9 | 35.7 |
| 0.17 | 60.2 | 2 | 40.3 | 53.7 | 37.7 | 43.2 | 48.4 | 55.9 |
| 0.25 | 78.4 | 3 | 52.9 | 69.3 | 48.2 | 56.4 | 63.5 | 72.1 |
| 0.33 | 90.9 | 4 | 65.8 | 82.5 | 55.6 | 66.6 | 77.7 | 83.0 |
| 0.50 | 98.5 | 6 | 82.9 | 93.9 | 70 | 80.5 | 93.2 | 94.3 |
| 0.75 | 100.6 | 8 | 93.3 | 98.0 | 79.9 | 88.1 | 100.3 | 98.5 |

The dissolution profiles of the composition of the present invention tested in 0.1N HCl indicate slower and sustained effect between 2 to 8 hrs as compared to Brand product, Plavix® which releases the drug almost completely within an hour.

Comparative dissolution profile of Plavix®, Example 10 & 11 in 900 ml 0.1N HCl using USP Type II apparatus at 50 rpm (FIG. 1):

The tablets of Example 4 were compared with Brand product Plavix® Clopidogrel bisulphate 75 mg tablets for anti-platelet aggregatory activity in platelet rich plasma from Beagle dogs ex-vivo.

The study was performed in 9-12 months old male dogs with body weight in the range 15-20 kg. Animals were fasted overnight. Single dose of Clopidogrel tablets of example 4 was administered to one group and the other group received Plavix®. Animals were bled at 0 (before drug administration) & 0.5, 2, 6, 10, 24, 48, 72, 96, 120, 168, 192 and 216 hr after drug administration. After 216 hr blood sampling a second dose of Clopidogrel was administered to both the groups and the animals were bled at 0 (216 hr after first dose) & 6, 24, 72, 240 hr after second dose administration.

Approximately 4 ml of Blood was collected in plastic syringes and transferred to plastic tubes tube containing 4% trisodium citrate solution.

(1:9::citrate solution:blood) and mixed gently with the anticoagulant.
Blood samples were centrifuged at 150 g for 10 min to obtain platelet rich plasma (PRP). After separation of PRP, the samples were centrifuged at 1500 g for 15 min to obtain platelet poor plasma (PPP). Platelet count of PRP was estimated using Cell-Dyne 3700 Hematology Analyzer and was adjusted to 3×10⁸/ml with PPP for ADP-induced aggregation assay.

180 μl of adjusted PRP of individual sample were incubated with 20 μL of ADP reagent (20 μM, final conc) was added and the optical density (OD) was monitored continuously every 30 seconds for 10 min at 560 nm using SPECTRAMAX 190 microplate reader. All samples were tested in triplicate.

### Data Analysis:

% Aggregation was calculated by using following formula:

$$\% Aggregation = \frac{Corrected O D ( 0 min ) - Corrected O D ( 10 min ) * 100}{Corrected O D ( 0 min )}$$

$$Corrected O D = Absorbance of P R P - Absorbance of P P P$$

Comparative data on % aggregation for Plavix and Clopidogrel tablets 75 mg as studied ex-vivo in beagle dogs is tabulated in table 3 and depicted in FIG. 2

TABLE 3
Effect of Clopidogrel Tablet formulation on
platelet aggregation, Ex-vivo study in beagle dogs

% Aggregation

| Treatment | Time (hr) | Plavix IR | Example 4 |
|---|---|---|---|
| First dose | 0 | 80.2 | 79.5 |
| | 0.5 | 34.9 | 49.4 |
| | 2 | 2.0 | 7.2 |
| | 6 | 3.6 | 3.0 |
| | 10 | 1.6 | 0.0 |
| | 24 | 3.2 | 0.0 |
| | 48 | 8.9 | 3.4 |
| | 72 | 17.9 | 9.6 |
| | 96 | 35.4 | 21.4 |
| | 120 | 53.0 | 32.1 |
| | 168 | 81.9 | 46.1 |
| | 192 | 86.1 | 61.2 |
| Second Dose | 0 (216 hr) | 80.2 | 63.0 |
| | 6 | −1.5 | −3.0 |
| | 24 | 4.4 | 4.9 |
| | 72 | 31.7 | 8.3 |
| | 120 | 57.6 | 21.8 |

Comparative platelet aggregation effect of Plavix, Clopidogrel Tablets 75 mg, Example 4, Ex-vivo study in Beagle dogs. (FIG. 2)

As shown in Table 3 and FIG. 2, all formulations of Clopidogrel caused significant inhibition of ADP induced platelet aggregation.

As compared to Plavix® tablets, Clopidogrel tablets of Example 4 showed effect sustained for longer period. The dog treated with Plavix® formulation showed complete reversal of the effect in ˜8-9 day, whereas the dog treated with Clopidogrel tablets of Example 4 showed anti-platelet aggregatory effect even on day 9. The second dose caused further reduction in platelet aggregation and effect for Clopidogrel tablets of Example 4 tablet was better than the Plavix® tablet.

Pharmacodynamic studies were performed with tablets prepared as per Example 10 as compared to Plavix® by determination of platelet functions in anticoagulated whole blood with the DiaMed Impact-R. The test method is aimed to study platelet function, screening of primary hemostasis abnormalities (e.g. Von Willebrand disease) including thrombocytopenia and monitoring their therapies. It can be used for testing both hypo- and hyperfunction of platelets. Furthermore, it provides a quick method for monitoring the response to various antiplatelet drugs. This test was studied to determine platelet functions in anticoagulated whole blood to compare Plavix® with modified release formulation modified release formulation of Clopidogrel, Example 10.

For the purpose of platelet aggregation assay, a total of 10 blood samples was collected.

The venous blood samples of 4.5 ml will be withdrawn at −10.00, 0.00 (pre dose) and 0.50, 2.00, 4.00, 6.00, 8.00, 10.00, 24.00 and 48.00 hours following drug administration during the study period.

Blood samples were collected through a fresh vein puncture, if it is fresh venipuncture 2-3 ml blood will be discarded because of possibilities of presence of some platelet aggregating factors due to fresh vein puncture. Blood samples were collected through an indwelling cannula placed in the forearm vein using disposable syringe. 4.5 ml blood sample will be withdrawn and transferred to sample collection tubes containing sodium citrate buffer at each sampling time point.

Whole blood samples to be tested was collected in tubes containing sodium citrate and stored at room temperature (18-25° C.). Citrated blood samples (130 μL) was placed in the centre of well and subjected to rotation process according to the pre-defined parameters. The well was washed and stained by May-Gruenwald stain.

Platelet adhesion was evaluated as the percentage of total area covered with platelets designated as surface coverage (%) and aggregation as the mean size of surface bound to aggregates designated as average size (μm²).

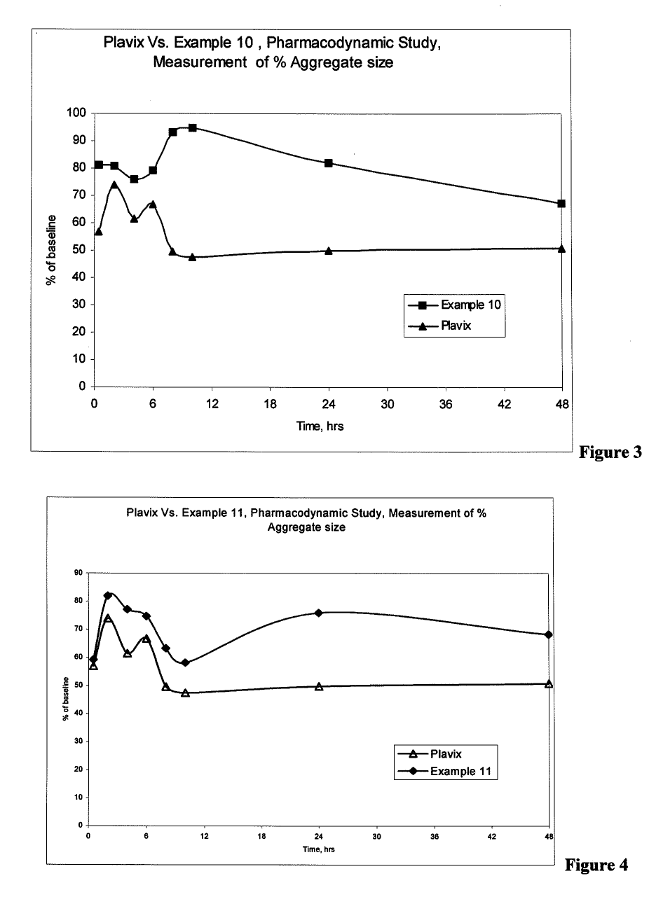

The comparative % aggregate size of platelets in the two formulations Plavix® Vs. Clopidogrel tablets, Example 10 and 11 is depicted in FIGS. 3 and 4.

The comparative % aggregate size of platelets in the two formulations Plavix® Vs. Clopidogrel tablets, Example 10 (FIG. 3).

Anti-platelet action was found to be better in Example 10 than Plavix® as reflected in the FIG. 3 as there is more de-aggregation of platelets in formulation of Example 10 as compared to Plavix®.

The comparative % aggregate size of platelets in the two formulations Plavix® Vs. Clopidogrel tablets, Example 11 (FIG. 4).

Anti-platelet action was found to be better in Example 11 than Plavix® as reflected in the FIG. 4 as there is more de-aggregation of platelets in formulation of Example 11 as compared to Plavix®.

Clopidogrel Besylate tablets equivalent to 75 mg Clopidogrel Example 10, Clopidogrel bisulphate tablets equivalent to 75 mg Clopidogrel Example 11 and Plavix®, marketed Clopidogrel bisulphate tablets were subjected to open label, balanced, randomized, three-treatment, single-period, single dose, parallel, comparative oral pharmacokinetic study in healthy, adult, male, human subjects under fasting conditions. The study was performed on 36 subjects (12 subjects for each treatment).

The subjects were housed at the clinical facility from not less than 11 hours pre-dose till 24-hour post-dose sample during the study period. The subjects were fasted overnight for at least 10 hours prior to dosing. Drinking water was prohibited from one hour before dosing till 2 hours post-dose except for dosing. At other times drinking water was provided at ad libitum. Meals were provided at around 4 hours after dosing and at specified intervals from then onwards till checkout in each period. A total of twenty-three blood samples were collected during the study period. The venous blood samples of 5.5 ml (including 0.5 ml discarded heparinised blood) were withdrawn at pre-dose (before dosing) and at 0.25, 0.50, 0.75, 1.00, 1.25, 1.50, 1.75, 2.00, 2.50, 3.00, 4.00, 5.00, 6.00, 7.00, 8.00, 9.00, 10.00, 12.00, 16.00, 24.00, 36.00 and 48.00 hours following drug administration during the study period. Blood samples were collected through an indwelling cannula placed in the forearm vein using disposable syringe. 5.5 ml blood sample (including 0.5 ml discarded heparinised blood) were withdrawn and transferred to sample collection tubes containing EDTA as anticoagulant at each sampling time point. After centrifugation, Plasma were separated with in one hour from blood samples and plasma samples were stored at −70±5° C. until withdrawn for analysis. Clopidogrel concentration in plasma were quantified using a validated LC-MS/MS.

Peak plasma concentration (Cₘₐₓ), time to reach peak plasma concentration (Tₘₐₓ), area under plasma concentration vs. time curve till the last time point (AUC₀₋ₜ), area under plasma concentration vs. time curve extrapolated to the infinity (AUC_{0-∞}), the residual area in percentage (AUC_% Extrap), the depuration (D), Volume of distribution (V_{d}), plasma elimination half-life (t_{1/2}) and elimination rate constant (λ_{z}), were calculated for Clopidogrel.

Mean plasma concentration at various time-points tested for Plavix®, Clopidogrel tablets prepared by Example 10 & 11 for Clopidogrel & Clopidogrel carboxy acid metabolite are tabulated in table 4 & 5 respectively. Mean values of pharmacokinetic parameters are tabulated in Table 6 & 7.

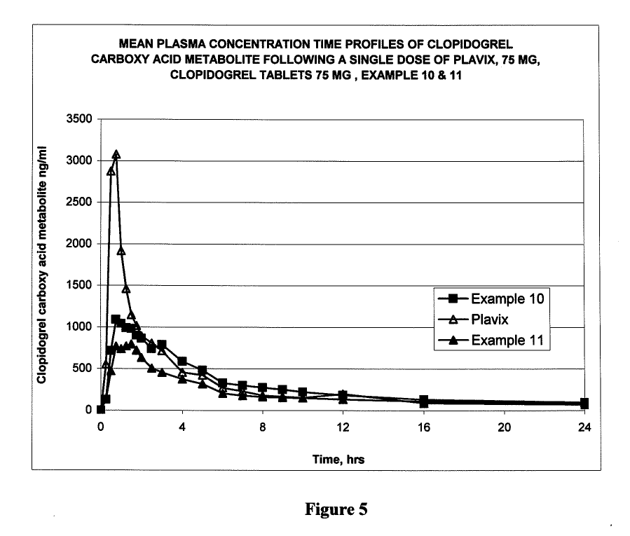

FIG. 5 depicts Mean Plasma concentration time profile of Clopidogrel carboxyacid metabolite (ng/mL) following a single dose of Plavix®, Clopidogrel tablets 75 mg, Example 10 and 11 under fasting conditions.

TABLE 4
Mean Plasma concentration time profile of
Clopidogrel (ng/mL) following a single dose of Plavix ®,
Clopidogrel tablets 75 mg, Example 10 & 11 under fasting
conditions.

| Time | Plavix ® | Example 10 | Example 11 |
|---|---|---|---|
| 0.00 | 0.000 | 0.000 | 0.000 |
| 0.25 | 0.221 | 0.000 | 0.036 |
| 0.50 | 1.021 | 0.457 | 0.231 |
| 0.75 | 0.889 | 0.571 | 0.313 |
| 1.00 | 0.816 | 0.662 | 0.338 |
| 1.50 | 0.751 | 0.571 | 1.084 |
| 2.00 | 0.333 | 0.603 | 1.234 |
| 2.50 | 0.059 | 0.785 | 0.156 |
| 3.00 | 0.076 | 0.865 | 0.110 |
| 4.00 | 0.068 | 0.590 | 0.000 |
| 6.00 | 0.000 | 0.454 | 0.000 |
| 8.00 | 0.000 | 0.402 | 0.000 |
| 10.00 | 0.000 | 0.278 | 0.000 |
| 12.00 | 0.000 | 0.099 | 0.000 |
| 24.00 | 0.000 | 0.095 | 0.000 |
| 36.15 | 0.000 | 0.000 | 0.000 |
| 49.17 | 0.000 | 0.000 | 0.000 |

TABLE 5
Mean Plasma concentration time profile of Clopidogrel
carboxyacid metabolite (ng/mL) following a single dose of
Plavix ®, Clopidogrel tablets 75 mg, Example 10 & 11 under
fasting conditions.

| Time | Plavix ® | Example 10 | Example 11 |
|---|---|---|---|
| 0.00 | 0.000 | 0.000 | 0.000 |
| 0.25 | 550.920 | 128.956 | 134.318 |
| 0.50 | 2873.385 | 716.962 | 471.477 |
| 0.75 | 3076.408 | 1089.771 | 769.039 |
| 1.00 | 1917.322 | 1042.220 | 737.130 |
| 1.25 | 1459.769 | 991.558 | 772.347 |
| 1.50 | 1145.545 | 980.296 | 794.055 |
| 1.75 | 1016.212 | 899.582 | 721.263 |
| 2.00 | 894.283 | 865.299 | 631.040 |
| 2.50 | 807.114 | 740.496 | 505.100 |
| 3.00 | 714.432 | 789.229 | 453.628 |
| 4.00 | 455.574 | 587.999 | 374.986 |
| 5.00 | 423.496 | 481.522 | 316.796 |
| 6.00 | 270.737 | 330.336 | 205.390 |
| 7.00 | 231.219 | 300.264 | 177.458 |
| 8.00 | 180.646 | 274.379 | 162.022 |
| 9.00 | 167.450 | 250.648 | 154.401 |
| 10.00 | 151.945 | 221.354 | 150.044 |
| 12.00 | 197.652 | 184.288 | 133.607 |
| 16.00 | 87.780 | 130.395 | 108.994 |
| 24.00 | 72.036 | 96.527 | 84.146 |
| 36.15 | 33.066 | 46.519 | 19.214 |
| 49.17 | 0.000 | 6.909 | 4.081 |

Mean Plasma concentration time profile of Clopidogrel carboxyacid metabolite (ng/mL) following a single dose of Plavix®, Clopidogrel tablets 75 mg, Example 10 & 11 under fasting conditions. (FIG. 5).

TABLE 6
Mean values of Pharmacokinetic parameters of
Clopidogrel following a single dose of Plavix ®, Clopidogrel
tablets 75 mg, Example 10 & 11 under fasting conditions.

| Variable | Plavix ® | Example 10 | Example 11 |
|---|---|---|---|
| Cmax (ng/ml) | 1.021 | 0.865 | 1.234 |
| AUC (ng/ml/h) | 1.590 | 5.904 | 1.592 |
| Tmax (Hrs) | 0.050 | 3.0 | 2.00 |

TABLE 7
Mean values of Pharmacokinetic parameters of
Clopidogrel carboxyacid metabolite following a single dose of
Plavix ®, Clopidogrel tablets 75 mg, Example 10 & 11 under
fasting conditions.

| Variable | Plavix ® | Example 10 | Example 11 |
|---|---|---|---|
| Cmax (ng/ml) | 3076.408 | 1089.771 | 794.055 |
| AUC (ng/ml/h) | 8503.204 | 8244.332 | 5716.368 |

Clopidogrel is a prodrug. The active metabolite, a thiol derivative, is formed by oxidation of Clopidogrel to 2-oxo-Clopidogrel and subsequent hydrolysis. The main circulating metabolite is Carboxy acid derivative, which is inactive. Rapid formation of carboxy acid derivative as observed by high plasma levels in Plavix® would correlate to exposure with high concentration of Clopidogrel and for a short period. In case of modified formulations of Clopidogrel of the present invention, Example 10, it can be seen that plasma concentration of Clopidogrel carboxy acid is in controlled and sustained manner between 2.5 to 8 hours. This would indicate slow formation of active metabolite which in turns converts slowly to carboxy acid derivative. It can be observed that formulations of the present invention show significantly lower Cmax for carboxy acid metabolite as compared to Brand product, Plavix®. The composition of the present invention shows higher AUC values for Clopidogrel as compared to Plavix®. Higher AUC values and greater Tmax for Clopidogrel along with significantly lower values of Cmax for inactive carboxy acid metabolite can be correlated to better availability of the drug for a longer duration of time.

While certain preferred and alternative embodiments of the invention have been set forth for purposes of disclosing the invention, modifications to the disclosed embodiments may occur to those who are skilled in the art. Accordingly, this specification is intended to cover all embodiments of the invention and modifications thereof that do not depart from the spirit and scope of the invention.

1. A once daily dosage form comprising Clopidogrel or a salt of Clopidogrel equivalent to 50 mg to 150 mg Clopidogrel, said dosage form providing at least one of the following in vivo plasma profile of Clopidogrel selected from:
(a) Mean Tmax of about 3 or more hours.
(b) Mean Cmax not above 1000 picogram/ml.
(c) Mean AUC₀₋₄₉ₕ of more than 2500 picogram/ml/hr.

2. A once daily dosage form comprising Clopidogrel or a salt of Clopidogrel equivalent to 50 mg to 150 mg Clopidogrel, said dosage form providing an in vivo plasma profile of Clopidogrel carboxy acid metabolite having Mean Cmax not above 1400 ng/ml.

3. The once daily dosage form as claimed in claim 1 wherein the dosage form comprises Clopidogrel or a salt of Clopidogrel equivalent to 75 mg Clopidogrel.

4. The once daily dosage form as claimed in claim 1, wherein the said mean Cmax is less than about 900 picogram/ml.

5. The once daily dosage form as claimed in claim 1, wherein the said mean Cmax is less than about 900 picogram/ml.

6. The once daily dosage form as claimed in claim 5, wherein the said mean Cmax is less than about 800 picogram/ml.

7. The once daily dosage form as claimed in claim 2, wherein the said mean Cmax is not above 1200 ng/ml.

8. The once daily dosage form as claimed in claim 2 wherein the dosage form comprises Clopidogrel or a salt of Clopidogrel equivalent to 75 mg Clopidogrel.

9. The once daily dosage form as claimed in claim 7, wherein the said mean Cmax is not more than 1100 ng/ml.
